(19) United States
(12) Patent Application Publication
(10) Pub. No.: US 2023/0346314 A1
(43) Pub. Date: Nov. 2, 2023

(54) Steerable Devices for Fiber Enabled Medical Systems

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 17/731,155

(22) Filed: Apr. 27, 2022

(51) Int. Cl. A61B 5/00

(52) U.S. Cl. CPC A61B 5/6852; A61B 2562/0266

(57) ABSTRACT

Embodiments disclosed herein are directed to steerable devices configured to negotiate tortuous vascular pathways. The system can include a stylet having a shapeable portion formed of a shape-memory, super-elastic material such as Nitinol. The stylet can be placed within a catheter and can include a fiber-optic strain sensor (FOSS) system configured to determine a shape of the stylet. Passing a fluid of a predetermined temperature through a lumen of the catheter can modify temperature of the shapeable portion which in turn can modify a shape and/or flexibility state of the shapeable portion. Modifying the shape of the shapeable portion can modify an angle at which a distal tip of the stylet extends relative to the central longitudinal axis. A user can then rotate and advance the distal tip to negotiate tortuous vascular pathways. Similarly, modifying a flexibility of the shapeable portion can facilitate negotiating tortuous vascular pathways.

### SUMMARY

Briefly summarized, embodiments of the present invention are directed to steerable devices for fiber enabled placement systems. The placement systems can be configured for tracking, placing, and monitoring an elongate medical device such as a stylet and/or catheter assembly inserted into a vasculature of a patient. The placement system utilizes optical fiber-based strain sensors to ascertain information regarding the elongate medical device during and/or after insertion into the patient's body. In one embodiment, the placement system comprises a steerable elongate medical device including a multi-core, fiber optic-based strain sensor (FOSS) system and a shapeable portion formed of a shape-memory, super-elastic material such as Nitinol.

The multi-core optical fiber can be configured to map a shape of the elongate medical device as it negotiates tortuous vascular pathways, to track a location of the elongate medical device. A laser light source (or other suitable light source) is also included and configured to operably connect with the strain sensors and produce outgoing optical signals incident on the strain sensors. A photodetector is included and configured to operably connect with the strain sensors and receive return optical signals from the strain sensors. A processor is configured to process data from the return optical signals. The data relates to an aspect of the medical device. A user interface such as a display is configured to communicate information relating to the aspect of the medical device.

The shapeable portion of the elongate medical device can be configured to transition between a first shape and a second shape in response to a change in temperature and facilitate steering a distal portion of the elongate medical device. For example, outside of the body at a relatively cooler temperature, the device can define a first, non-linear shape. Inside the body at a relatively warmer temperature, the device can define a second, linear shape. Alternatively, the first shape can define a linear shape and the second shape can define a non-linear shape. Passing fluids of different temperatures over, or through, the elongate medical device can modify the shape of the shapeable portion between the first shape and the second shape, or to a transitional shape therebetween. In an embodiment, the shapeable portion can transition between a relatively rigid state and a relatively flexible state in response to a change in temperature. As such, a user can selectively modify the shape and/or flexibility of the elongate medical device to facilitate steering the device through tortuous vasculature pathways.

Disclosed herein is fiber-optic enabled intravascular system including, a catheter defining a lumen extending along a central longitudinal axis, an elongate medical device disposed within the catheter lumen and including a shapeable portion, the shapeable portion including a shape-memory, or super-elastic material, the shapeable portion defining one or both of a first shape and a first flexibility state at a first temperature and one or both of a second shape and a second flexibility state at a second temperature, which is greater than the first temperature.

In some embodiments, the fiber-optic enabled intravascular system further includes a steering control system configured to provide a fluid to the catheter lumen and modify a temperature of the fluid to modify a temperature of the shapeable portion between the first temperature and the second temperature.

In some embodiments, the steering control system includes one or more of a handle, a fluid source, a pump, and a temperature regulation device configured to modify the temperature of the fluid.

In some embodiments, the temperature regulation device includes one or more of heat source, a cooling source, a thermoelectric generator, a Seebeck generator, a heat pump, a refrigeration system, an immersion heater, an induction heater, and an infrared heater.

In some embodiments, the elongate medical device includes one of a stylet, trocar, guidewire, or catheter.

In some embodiments, the elongate medical device is formed of a first material and the shapeable portion is formed of a second material, the first material including a plastic, polymer, metal, alloy, or composite, the second material including a metal, alloy, shape-memory material, super-elastic material, or Nitinol.

In some embodiments, the elongate medical device further includes an optical fiber extending longitudinally and communicatively coupled to a fiber optic strain sensor system configured to determine a shape of the elongate medical device.

In some embodiments, the shapeable portion extends annularly about a portion of the optical fiber.

In some embodiments, the shapeable portion is disposed distally of a distal tip of the optical fiber.

In some embodiments, the fiber-optic enabled intravascular system further includes one or both of a transition shape and a transition flexibility state at a third temperature, which is between the first temperature and the second temperature.

In some embodiments, the first shape is a linear shape and the second shape is a non-linear shape.

In some embodiments, the first shape is a non-linear shape and the second shape is a linear shape.

In some embodiments, the first shape is a linear shape and the second shape is a curved shape where an axis of a distal tip of the elongate medical device extends at a first angle relative to the central longitudinal axis, and a transition shape is a curved shape where an axis of the distal tip of the elongate medical device extends at a second angle relative to the central longitudinal axis, less than the first angle.

In some embodiments, the elongate medical device further includes a first lumen, and wherein the steering control system is in fluid communication with the first lumen and configured to modify a temperature of the fluid within the first lumen.

In some embodiments, the elongate medical device further includes a second lumen, and wherein the steering control system is configured to modify a temperature of the fluid within the first lumen independently of a temperature of the fluid within the second lumen.

Also disclosed is a method of placing a catheter within a vasculature including, advancing a distal tip of an elongate medical device into a vasculature of a patient, the elongate medical device including a shapeable portion and disposed within a lumen of a catheter extending along a central longitudinal axis, modifying a temperature of a fluid to a first predetermined temperature, urging the fluid through the catheter lumen, modifying a temperature of the shapeable portion, and modifying one or both of a shape and a flexibility state of the shapeable portion.

In some embodiments, the step of modifying the temperature of the fluid includes modifying one or both of a pump and a temperature regulation device of a steering control system, the temperature regulation device including one of a heat source, a cooling source, a thermoelectric generator, a Seebeck generator, a heat pump, a refrigeration system, an immersion heater, an induction heater, and an infrared heater.

In some embodiments, the elongate medical device includes one of a stylet, trocar, guidewire, or catheter.

In some embodiments, the elongate medical device is formed of a first material and the shapeable portion is formed of a second material, the first material including a plastic, polymer, metal, alloy, or composite, the second material including a metal, alloy, shape-memory material, super-elastic material, or Nitinol.

In some embodiments, the shapeable portion transitions between a first shape at a first temperature, and a second shape at a second temperature, the second temperature being greater than the first temperature, and a transition shape at a third temperature that is between the first temperature and the second temperature.

In some embodiments, the first shape is a linear shape and the second shape is a curved shape where an axis of a distal tip of the elongate medical device extends at a first angle relative to the central longitudinal axis, and a transition shape is a curved shape where an axis of the distal tip of the elongate medical device extends at a second angle relative to the central longitudinal axis, less than the first angle.

In some embodiments, the shapeable portion transitions between a first flexibility state at a first temperature, and a second flexibility state at a second temperature, the second temperature being greater than the first temperature, and a transition flexibility state at a third temperature that is between the first temperature and the second temperature.

In some embodiments, the elongate medical device further includes an optical fiber extending therethrough and communicatively coupled to a fiber optic strain sensor system configured to determine a shape of the elongate medical device.

Also disclosed is a method of placing a fiber-optic enabled medical device within a vasculature including, providing a the fiber-optic enabled medical device including a shapeable portion at a first temperature, the shapeable portion formed of a shape-memory material, advancing a distal tip of the fiber-optic enabled medical device into a vasculature of a patient, modifying a temperature of the shapeable portion from the first temperature to a second temperature, greater than the first temperature, and modifying one or both of a shape and a flexibility state of the shapeable portion.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a schematic view of a fiber optic-based strain sensor (FOSS) system, in accordance with embodiments disclosed herein.

FIG. 2 shows a perspective view of a stylet assembly for use with the system of FIG. 1, in accordance with embodiments disclosed herein.

FIG. 3 shows a perspective view of a stylet and catheter assembly for use with the system of FIG. 1, in accordance with embodiments disclosed herein.

FIG. 4 shows the system of FIGS. 1-3 including a stylet and catheter assembly inserted into a vasculature of a patient, in accordance with embodiments disclosed herein.

FIG. 5 shows an exemplary structure of a section of the multi-core optical fiber included within the stylet assembly of FIGS. 1-3, in accordance with embodiments disclosed herein.

FIG. 6A shows a perspective view of a multi-core optical fiber included within an elongate medical device, in accordance with embodiments disclosed herein.

FIG. 6B shows a cross-section view of the optical fiber of FIG. 6A, in accordance with embodiments disclosed herein.

FIG. 7A shows close up detail of a distal portion of an elongate medical device including a shapeable portion in a linear shape, in accordance with embodiments disclosed herein.

FIG. 7B shows close up detail of a distal portion of an elongate medical device including a shapeable portion in a non-linear shape, in accordance with embodiments disclosed herein.

FIG. 8 shows close up detail of a distal portion of an elongate medical device including a shapeable portion in a linear shape, in accordance with embodiments disclosed herein.

FIG. 9 shows close up detail of a distal portion of an elongate medical device including a shapeable portion in a linear shape, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention, and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

The term “logic” is representative of hardware and/or software that is configured to perform one or more functions. As hardware, logic may include circuitry having data processing and/or storage functionality. Examples of such circuitry may include, but are not limited or restricted to a processor, a programmable gate array, a microcontroller, an application specific integrated circuit, combinatorial circuitry, or the like. Alternatively, or in combination with the hardware circuitry described above, the logic may be software in the form of one or more software modules, which may be configured to operate as its counterpart circuitry. The software modules may include, for example, an executable application, a daemon application, an application programming interface (API), a subroutine, a function, a procedure, a routine, source code, or even one or more instructions. The software module(s) may be stored in any type of a suitable non-transitory storage medium, such as a programmable circuit, a semiconductor memory, non-persistent storage such as volatile memory (e.g., any type of random access memory “RAM”), persistent storage such as non-volatile memory (e.g., read-only memory “ROM”, power-backed RAM, flash memory, phase-change memory, etc.), a solid-state drive, hard disk drive, an optical disc drive, or a portable memory device.

With respect to “proximal,” a “proximal portion” or a “proximal end portion” of, for example, a stylet disclosed herein includes a portion of the stylet intended to be near a clinician when the stylet is used on a patient. Likewise, a “proximal length” of, for example, the stylet includes a length of the stylet intended to be near the clinician when the stylet is used on the patient. A “proximal end” of, for example, the stylet includes an end of the stylet intended to be near the clinician when the stylet is used on the patient. The proximal portion, the proximal end portion, or the proximal length of the stylet can include the proximal end of the stylet; however, the proximal portion, the proximal end portion, or the proximal length of the stylet need not include the proximal end of the stylet. That is, unless context suggests otherwise, the proximal portion, the proximal end portion, or the proximal length of the stylet is not a terminal portion or terminal length of the stylet.

With respect to “distal,” a “distal portion” or a “distal end portion” of, for example, a stylet disclosed herein includes a portion of the stylet intended to be near or in a patient when the stylet is used on the patient. Likewise, a “distal length” of, for example, the stylet includes a length of the stylet intended to be near or in the patient when the stylet is used on the patient. A “distal end” of, for example, the stylet includes an end of the stylet intended to be near or in the patient when the stylet is used on the patient. The distal portion, the distal end portion, or the distal length of the stylet can include the distal end of the stylet; however, the distal portion, the distal end portion, or the distal length of the stylet need not include the distal end of the stylet. That is, unless context suggests otherwise, the distal portion, the distal end portion, or the distal length of the stylet is not a terminal portion or terminal length of the stylet.

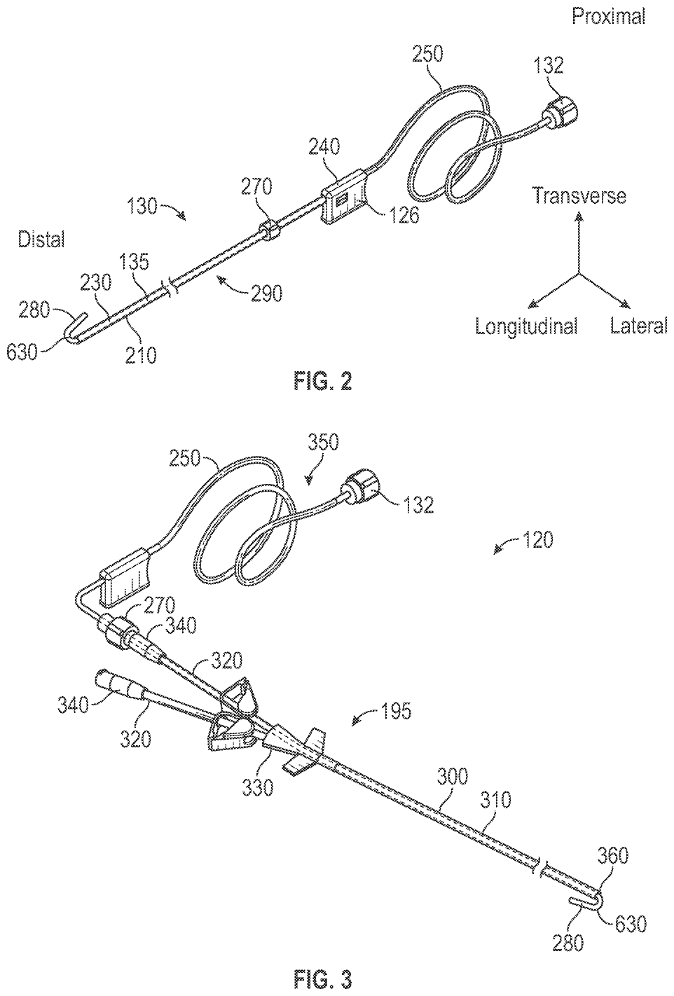

To assist in the description of embodiments described herein, as shown in FIG. 2, a longitudinal axis extends substantially parallel to an axial length of the stylet. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

It is important to note that, though the below discussion focuses on usage of a stylet for the placement of a catheter into the body of the patient, the stylet described herein can be employed to place a variety of medical devices, especially other elongate medical devices, in a variety of locations within the patient body. As such, the principles of the present disclosure should not be considered limiting to what is explicitly described herein. Examples of catheter assemblies and medical devices that may benefit from the disclosure may include a peripherally inserted central catheter (“PICC”), central venous catheter (“CVC”), urinary catheter, midline catheter, peripheral catheter, or the like.

In light of the above, a multi-core optical fiber can also be paired with one or more conductive medium for electrical signal monitoring thus serves multiple modalities. For example, the first modality constitutes an optical modality with shape sensing functionality to determine the physical state of the stylet, or similar elongate medical device. The physical state of the stylet provides information to assist a clinician in guiding a catheter assembly to a desired location within the vasculature.

The one or more second modalities can include but not limited to a tip location/navigation system (“TLS”) modality and/or an ECG modality. In an embodiment, a tip location/navigation system (“TLS”) modality includes where the stylet with conductive medium is advanced to detect and avoid any tip malposition during such advancement. In an embodiment, an ECG modality includes wherein ECG signal-based catheter tip guidance is employed to enable tracking and guidance of the stylet/catheter tip to a desired position with respect to a node of the patient's heart from which the ECG signals originate.

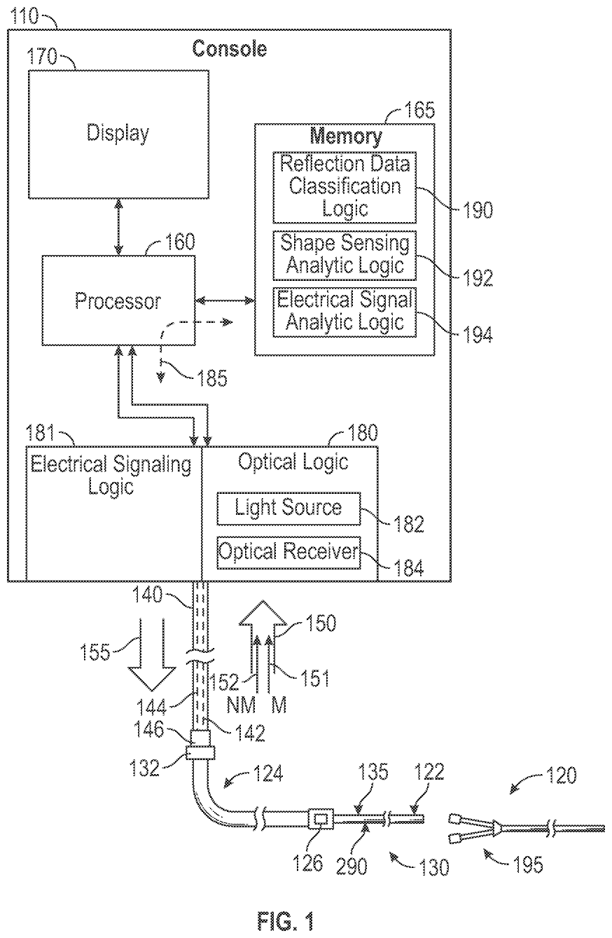

Referring to FIG. 1, an illustrative embodiment of a medical device monitoring system (“system”) 100 is shown. As shown, the system 100 generally includes a console 110 and a handheld, elongate medical device such as a stylet/catheter assembly 120 communicatively coupled to the console 110. It will be appreciated, however, that the stylet/catheter assembly 120 is exemplary and the assembly 120 can include various stylets, trocars, guidewires, catheters, or combinations thereof. For this embodiment, the stylet/catheter assembly 120 includes a stylet assembly 130 coupled with a catheter assembly 195, as described in more detail herein.

In an embodiment, the stylet assembly 130 includes an elongate probe (e.g., stylet body) 290 on its distal end 122 and a console connector 132 on its proximal end 124. The console connector 132 enables the stylet assembly 130 to be operably connected to the console 110 via an interconnect 140 including one or more optical fibers 142 (hereinafter, “optical fiber(s)”) and, optionally, a conductive medium 144 terminated by one or more optical/electric connectors (“connector”) 146. Herein, the connector 146 is configured to engage (mate) with the console connector 132 to allow for the propagation of light between the console 110 and the stylet assembly 130 as well as the propagation of electrical signals from the stylet 290 to the console 110.

An exemplary implementation of the console 110 includes a processor 160, a memory 165, a display 170 and one or more logic engines such as an optical logic 180, an electrical signaling logic 181, a reflection data classification logic 190, a shape sensing analytic logic 192, and an electrical signal analytic logic 194. Although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The processor 160, with access to the memory 165 (e.g., non-volatile memory), is included to control functionality of the console 110 during operation. As shown, the display 165 may be a liquid crystal diode (LCD) display integrated into the console 110 and employed as a user interface to display information to the clinician, especially during a catheter placement procedure (e.g., cardiac catheterization). In another embodiment, the display 165 may be separate from the console 110. Although not shown, a user interface is configured to provide user control of the console 110.

For both of these embodiments, the content depicted by the display 165 may change according to which mode the stylet 290 is configured to operate, e.g. optical, TLS, ECG, or other modality. In TLS mode, the content rendered by the display 165 may constitute a two-dimensional (2-D) or three-dimensional (3-D) representation of the physical state (e.g., length, shape, form, and/or orientation) of the stylet 290 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below.

According to one embodiment of the disclosure, an activation control 126, included on the stylet assembly 130, may be used to set the stylet 290 into a desired operating mode and selectively alter operability the display 165 by the clinician to assist in medical device placement. For example, based on the modality of the stylet 290, the display 165 of the console 110 can be employed for optical modality-based guidance during catheter advancement through the vasculature or TLS modality to determine the physical state (e.g., length, form, shape, orientation, etc.) of the stylet 290. In one embodiment, information from multiple modes, such as optical, TLS, ECG, etc., may be displayed concurrently (e.g., at least partially overlapping in time). In one embodiment, the display 165 is a liquid crystal diode (LCD) device or a touch screen device.

Referring still to FIG. 1, the optical logic 180 is configured to support operability of the stylet assembly 130 and enable the return of information to the console 110, which may be used to determine the physical state associated with the stylet 290 along with monitored electrical signals, such as ECG signaling, electrophysiological sensors, etc., via an electrical signaling logic 181 that supports receipt and processing of the received electrical signals from the stylet 290 (e.g., ports, analog-to-digital conversion logic, etc.). The physical state of the stylet 290 may be based on changes in characteristics of the reflected light signals 150 received from the stylet 290. The characteristics may include shifts in wavelength caused by strain on certain regions of the core fibers integrated within a multi-core optical fiber 135 positioned within or operating as the stylet 290, as shown below. From information associated with the reflected light signals 150, the console 110 may determine (through computation or extrapolation of the wavelength shifts) the physical state of the stylet 290, and notably a catheter assembly 195 configured to receive the stylet 290.

According to one embodiment of the disclosure, as shown in FIG. 1, the optical logic 180 may include a light source 182 and an optical receiver 184. The light source 182 is configured to transmit the broadband incident light 155 for propagation over the optical fiber(s) 142 included in the interconnect 140, which are optically connected to the multi-core optical fiber 135 within the stylet 290. In one embodiment, the light source 182 is a tunable swept laser, although other suitable light source can also be employed in addition to a laser, including semi-coherent light sources, LED light sources, etc.

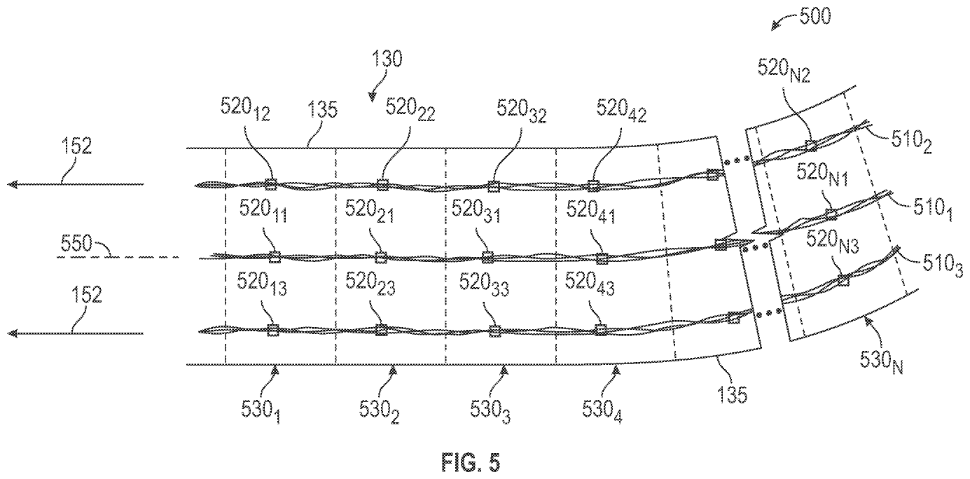

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 290 (see FIGS. 2 and 5), and (ii) translate the reflected light signals 150 into reflection data 185, namely data in the form of electrical signals representative of the reflected light signals including wavelength shifts caused by strain. The reflected light signals 150 associated with different spectral widths may include reflected light signals 151 provided from sensors positioned in the center core fiber, e.g. center core fiber 510₁, of the multi-core optical fiber 135 and reflected light signals 152 provided from sensors positioned in the periphery core fibers, e.g. periphery core fibers 510₁₋₄, of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data 185 to the memory 165 for storage and processing by reflection data classification logic 190. The reflection data classification logic 190 may be configured to: (i) identify which core fibers pertain to which of the received reflection data 185 and (ii) segregate the reflection data 185 provided from reflected light signals 150 pertaining to similar regions of the stylet 290 or spectral widths into analysis groups. The reflection data for each analysis group is made available to shape sensing analytic logic 192 for analytics.

According to one embodiment of the disclosure, the shape sensing analytic logic 192 is configured to compare wavelength shifts measured by sensors deployed in each periphery core fiber at the same measurement region of the stylet 290 (or same spectral width) to the wavelength shift at a center core fiber of the multi-core optical fiber 135 positioned along central axis and operating as a neutral axis of bending. From these analytics, the shape sensing analytic logic 192 may determine the shape the core fibers have taken in 3-D space and may further determine the current physical state of the catheter assembly 195 in 3-D space for rendering on the display 170.

According to one embodiment of the disclosure, the shape sensing analytic logic 192 may generate a rendering of the current physical state of the stylet 290 (and potentially the catheter assembly 195), based on heuristics or run-time analytics. For example, the shape sensing analytic logic 192 may be configured in accordance with machine-learning techniques to access a data store (library) with pre-stored data (e.g., images, etc.) pertaining to different regions of the stylet 290 (or catheter assembly 195) in which reflected light from core fibers have previously experienced similar or identical wavelength shifts. From the pre-stored data, the current physical state of the stylet 290 (or catheter assembly 195) may be rendered. Alternatively, as another example, the shape sensing analytic logic 192 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 290 (and/or catheter assembly 195), especially to enable guidance of the stylet 290, when positioned at a distal tip of the catheter assembly 195, within the vasculature of the patient and at a desired destination within the body.

The console 110 may further include electrical signal receiver logic 186, which is positioned to receive one or more electrical signals from the stylet 290. In an embodiment, the stylet 290 is configured to support both optical connectivity as well as electrical connectivity. The electrical signal receiver logic 186 is configured to send or receive the electrical signals or electrical energy to/from the stylet 290 via the conductive medium 144/230.

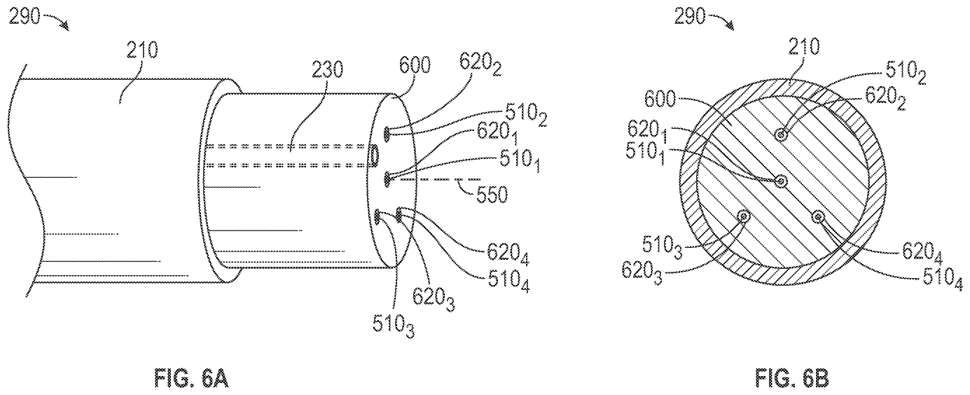

Referring now to FIG. 2, an exemplary embodiment of the stylet assembly 130 to be operably connected to the catheter assembly 195 (FIG. 3) is shown. Herein, the stylet assembly 130 features the stylet 290, which includes an insulating layer 210 encasing a multi-core optical fiber 135 and/or conductive medium 230 (e.g. FIGS. 6A-6B). The stylet 290 extends distally from a handle 240 while an interconnect (e.g. tether) 250 extends proximally from the handle 240 and is terminated by the console connector 132 for coupling to the interconnect 140 of the console 110 as shown in FIG. 1. The handle 240 is included with the second interconnect (e.g., tether) 250 to assist with manipulation of the stylet 290 by the user during operation and may be configured to include activation controls 126.

As shown, the stylet 290 and the interconnect 250 provide a pathway for outgoing optical signals produced by the light source 182 of the optical logic 180 and returning optical signals, produced by gratings within the core fibers of the multi-core optical fiber 135, for receipt by the photodetector 184 (see FIG. 1). Insulating layers associated with the stylet 290 and the interconnect 250 may vary in density and material to control its rigidity and mechanical properties, as described herein.

Furthermore, according to one embodiment of the disclosure, the stylet assembly 130 further includes a catheter connector 270, which may be threaded for attachment to a connector of an extension leg of a catheter assembly 195 (see FIG. 3). This connectivity between the connector 270 and a connector of the extension leg connector may be used during the procedure of inserting the stylet 290 into a lumen of the catheter assembly 195, as shown in FIG. 3. When deployed, a distal end of the multi-core optical fiber 135 need not be substantially co-terminal with a distal tip 360 of the catheter assembly 195. As will be seen, the returned optical signals (reflected light 150) from the sensors (reflective gratings) within each core fiber included with the multi-core optical fiber 135 may be analyzed during its advancement through the patient vasculature.

Note further that, it should appreciated that the term “stylet,” as used herein, can include any one of a variety of devices configured for removable placement within a lumen of the catheter (or other portion of a medical device) to assist in placing a distal end of the catheter in a desired location within the patient's vasculature. Also, note that other connection schemes between the stylet 290 and the console 110 can also be used without limitation.

Referring to FIG. 3, an embodiment of the stylet assembly 130 for placement within the catheter assembly 195 to provide a stylet/catheter assembly 120 is shown. Herein, the catheter assembly 195 includes an elongate catheter tube 300 defining one or more lumens 310 extending between proximal and distal ends of the catheter tube 300. The catheter tube 300 is in communication with a corresponding extension leg 320 via a bifurcation hub 330. Luer connectors 340 are included on the proximal ends of the extension legs 320.

As shown, the stylet assembly 130 includes the console connector 132 on its proximal end 350 to enable the stylet 290 to operably connect with the console 110 (see FIG. 1). The interconnect 250 distally extends communications from the console 110 to the catheter connector 270, which is configured to threadably engage (or otherwise connect with) the Luer connector 340 of one of the extension legs 320 of the catheter assembly 195. The stylet 290 extends distally from the catheter connector 270 up to a distal-end 280 of the stylet 290. The distal-end 280 of the stylet 290 may be substantially co-terminal with a distal tip 360 of the catheter assembly 195 within the vasculature.

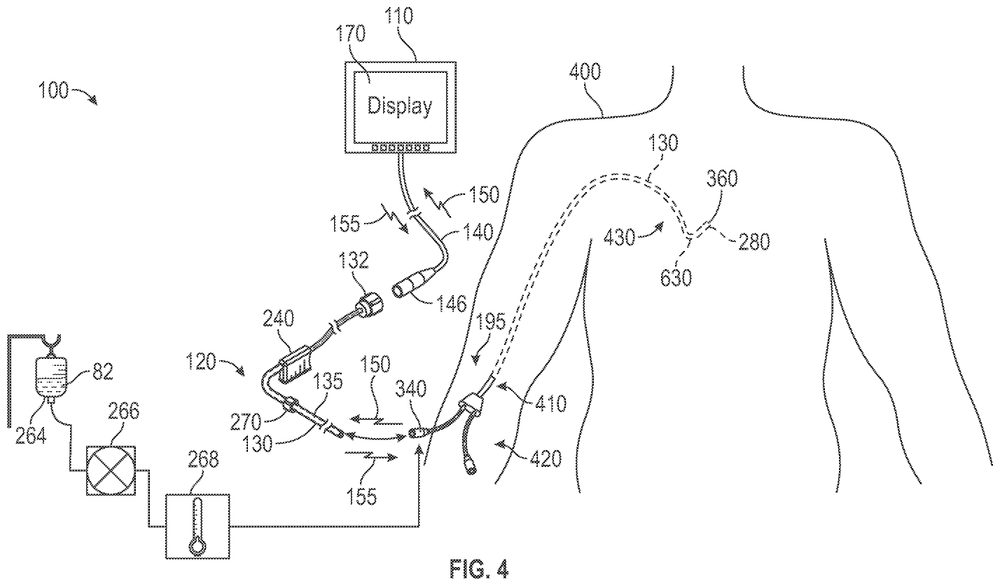

Referring now to FIG. 4, an embodiment of the stylet 290 illustrating its placement within the catheter assembly 195 as the catheter assembly 195 is being inserted into a vasculature of a patient 400 through a skin insertion site 410 is shown. As illustrated in FIG. 4, the catheter assembly 195 generally includes a proximal portion 420 that generally remains exterior to the patient 400 and a distal portion 430 that generally resides within the patient vasculature after placement is complete. The stylet 290 is employed to assist in the positioning of the distal tip 360 of the catheter assembly 195 in a desired position within the patient vasculature. In one embodiment, the desired position for the catheter distal tip 360 is proximate the patient's heart, such as in the lower one-third (⅓^{rd}) portion of the Superior Vena Cava (“SVC”) for this embodiment. Of course, the stylet 290 can be employed to place the catheter distal tip 360 in other locations.

During advancement of the catheter assembly 195, the stylet 290 receives broadband light 155 from the console 110 via interconnect 140, which includes the connector 146 for coupling to the console connector 132 for the stylet assembly 130. The reflected light 150 from sensors (reflective gratings) within each core fiber of the multi-core optical fiber 135 are returned from the stylet 290 over the interconnect 140 for processing by the console 110. The physical state of the stylet 290 may be ascertained based on analytics of the wavelength shifts of the reflected light 150. For example, the strain caused through bending of the stylet 290, and hence angular modification of each core fiber, causes different degrees of deformation. The different degrees of deformation alters the shape of the sensors (reflective grating) positioned on the core fiber, which may cause variations (shifts) in the wavelength of the reflected light from the sensors positioned on each core fiber within the multi-core optical fiber 135, as shown in FIG. 5. From this wavelength shifting, the shape sensing analytic logic 192 within the console 110 (see FIG. 1) may determine the physical state of the stylet 290 (e.g., shape, orientation, etc.).

Referring to FIG. 5, an exemplary embodiment of a right-sided, longitudinal view of a section 500 of the multi-core optical fiber 135 included within the stylet 290 is shown. The multi-core optical fiber section 500 depicts certain core fibers 510₁-510_{M} (M≥2, M=4 as shown) along with the spatial relationship between sensors (e.g., reflective gratings) 520₁₁-520_{NM} (N≥2; M≥2) present within the core fibers 510₁-510_{M}, respectively. As shown, the section 500 is subdivided into a plurality of cross-sectional regions 530₁-530_{N}, where each cross-sectional region 530₁-530_{N} corresponds to reflective gratings 520₁₁-520₁₄ . . . 520_{N1}-520_{N4}. Some or all of the cross-sectional regions 530₁ . . . 530_{N} may be static (e.g., prescribed length) or may be dynamic (e.g., vary in size among the regions 530₁ . . . 530_{N}). A first core fiber 510₁ is positioned substantially along a center (neutral) axis 550 while core fiber 510₂ may be oriented within the cladding of the multi-core optical fiber 135, from a cross-sectional, front-facing perspective, to be position on “top” the first core fiber 510₁. In this deployment, the core fibers 510₃ and 510₄ may be positioned “bottom left” and “bottom right” of the first core fiber 510₁. (See FIG. 6B).

Referencing the first core fiber 510₁ as an illustrative example, when the stylet 290 is operative, each of the reflective gratings 520₁-520_{N} reflect light for a different spectral width. As shown, each of the gratings 520₁ᵢ-520_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 510₂-510₃ but along at the same cross-sectional regions 530-530_{N} of the multi-core optical fiber 135, the gratings 520₁₂-520_{N2} and 520₁₃-520_{N3} are configured to reflect incoming light at same (or substantially similar) center frequency. As a result, the reflected light returns information that allows for a determination of the physical state of the optical fiber 135 (and the stylet 290) based on wavelength shifts measured from the returned, reflected light. In particular, strain (e.g., compression or tension) applied to the multi-core optical fiber 135 (e.g., at least core fibers 510₂-510₃) results in wavelength shifts associated with the returned, reflected light. Based on different locations, the core fibers 510₁-510₄ experience different types and degree of strain based on angular path changes as the stylet 290 advances in the patient.

For example, with respect to the multi-core optical fiber section 500 of FIG. 5, in response to angular (e.g., radial) movement of the stylet 290 is in the left-veering direction, the second core fiber 510₂ of the multi-core optical fiber 135 with the shortest radius during movement (e.g., core fiber closest to a direction of angular change) would exhibit compression (e.g., forces to shorten length). At the same time, the third core fiber 510₃ with the longest radius during movement (e.g., core fiber furthest from the direction of angular change) would exhibit tension (e.g., forces to increase length). As these forces are different and unequal, the reflected light from reflective gratings 520_{N2} and 520_{N3} associated with the core fiber 510₂ and 510₃ will exhibit different changes in wavelength. The differences in wavelength shift of the reflected light signals 152 can be used to extrapolate the physical configuration of the stylet 290 by determining the degrees of wavelength change caused by compression/tension for each of the periphery fibers (e.g., the second core fiber 510₂, the third core fiber 510₃, and the fourth core fiber 510₄) in comparison to the wavelength of the reference core fiber (e.g., first core fiber 510₁) located along the neutral axis 550 of the multi-core optical fiber 135. These degrees of wavelength change may be used to extrapolate the physical state of the stylet 290.

Referring now to FIG. 6A, an exemplary embodiment of a multimodal stylet 290 of FIG. 1 supporting one or both of an optical and electrical signaling is shown. Herein, the stylet 290 features a centrally located multi-core optical fiber 135, which includes a cladding 600 and a plurality of core fibers 510₁-510_{M} (M≥2; M=4) residing within a corresponding plurality of lumens 620₁-620_{M}. While the multi-core optical fiber 135 is illustrated within four (4) core fibers 510₁-510₄, a greater number of core fibers 510₁-510_{M} (M>4) may be deployed to provide a more detailed three-dimensional sensing of the physical state (e.g., shape, orientation, etc.) of the multi-core optical fiber 135 and the stylet 290 deploying the optical fiber 135, a greater number of core fibers 510₁-510_{M} (M>4) may be deployed. In an embodiment, the optical fiber 135 can include an outer insulating layer 210 disposed on an outer surface thereof. In an embodiment, the stylet 290 can include a conductive medium 230 extending therethrough to provide an electrically conductive pathway therethrough.

Further details, examples and embodiments of fiber-optic enabled strain sensor (FOSS) systems can be found in U.S. 2018/0289927, U.S. 2021/0045814, U.S. 2021/0156676, U.S. 2021/0154440, U.S. 2021/0275257, U.S. 2021/0268229, U.S. 2021/0271035, U.S. 2021/0402144, U.S. 2021/0401509, U.S. 2022/0011192, and U.S. 2022/0034733, each of which are incorporated by reference in their entirety.

In further reference to FIG. 4, in an embodiment, the elongate medical device 130 can include a shapeable portion 630 formed of a shape-memory or super-elastic material such as Nitinol. In an embodiment, the system 100 can further include a steering control system 260 which can include one or more of a fluid source 264, a pump 266, and a temperature regulation device 268. The fluid source 264 can include a syringe, fluid bag, fluid line, or the like configured to provide an infusate fluid 82 to lumen 310 of the catheter assembly 195. The pump 266 can include a syringe, mechanical pump, electro-mechanical pump, hand operated pump, gravity fed pump, or similar device configured to urge the fluid 82 through the catheter lumen 310. The temperature regulation device 268 can include a device configured to warm or cool the fluid 82 to a predetermined temperature prior to entering the catheter lumen 310.

In an embodiment, the steering control system 260 can be configured to modify one or both of a temperature and a flow rate of fluid 82 passing over, or through, a shapeable portion 630 of the stylet assembly 130 to modify a shape and/or flexibility of the shapeable portion 630. The stylet 290 can be further manipulated by manipulating the handle 240, either by a longitudinal movement or by a rotational movement about a central axis 550 of the stylet assembly 130. As such, a user can steer a distal portion of the stylet assembly 130 through the tortuous pathways of the vasculature of the patient 400 to a target location.

As will be appreciated, the stylet 290 is not intended to limiting and various elongate medical devices such as guidewires, trocars, catheters, or the like, can be used in like manner. As shown in FIG. 3, in an embodiment, the stylet 290 be disposed within a lumen of one or more elongate medical devices such as a catheter assembly 195. In an embodiment, the elongate medical device 130 can define a lumen 134 in fluid communication with the steering control system 260 and configured to receive a temperature regulated infusate fluid 82 therethrough.

In an embodiment, the stylet 290 can be formed of one or more materials including, but not limited to, a plastic, polymer, elastomer, thermoplastic, metal, alloy, shape-memory material, super-elastic material, Nitinol, composite, or combinations thereof. In an embodiment, the stylet 290 can include a first material, as described herein, and a portion of the stylet 290 can include a second material including but not limited to, a plastic, polymer, elastomer, thermoplastic, metal, alloy, shape-memory material, super-elastic material, Nitinol, composite, or combinations thereof.

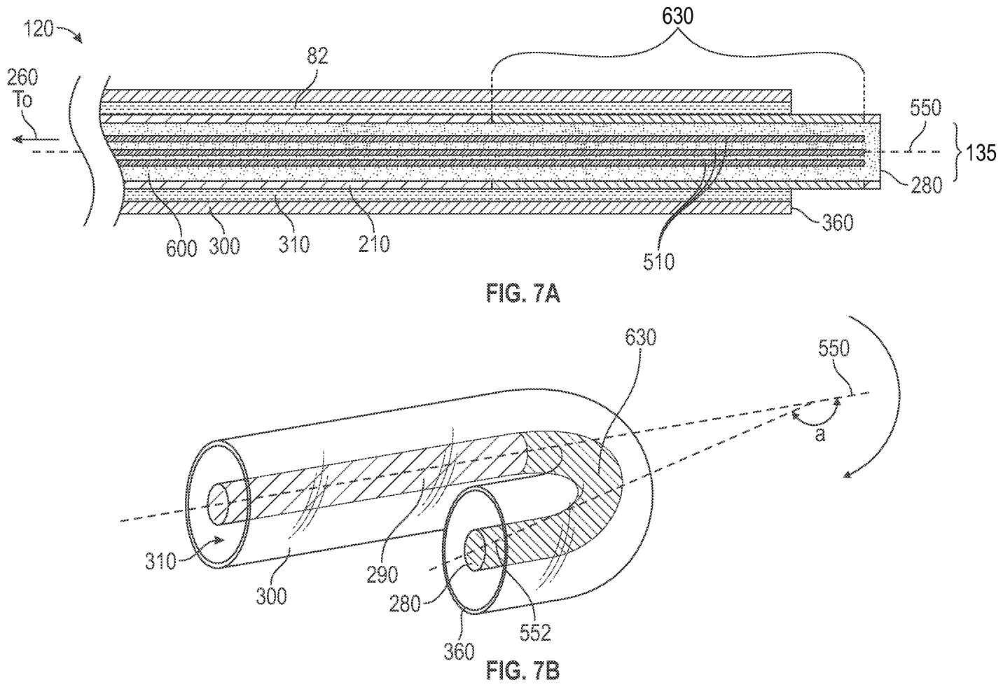

As shown in FIGS. 7A-7B, in an embodiment, the stylet 290 can include one or more shapeable portions 630 that include a shape-memory or super-elastic material such as Nitinol, or the like. In an embodiment, the shapeable portion 630 can be configured to be selectively shapeable between a first shape and a second shape, or to a transitional shape therebetween. In an embodiment, the shapeable portion 630 can be configured to be selectively transitionable between a relatively rigid state and a relatively flexible state.

In an embodiment, the shapeable portion 630 can define a first shape at a first temperature, and a second shape different from the first shape, at a second temperature. The first temperature can be lower relative to the second temperature. In an embodiment, the first temperature can be less than 37° C. (98.6° F.) and the second temperature can be equal to or greater than 37° C. (98.6° F.). It will be appreciated, however, that these temperature ranges are exemplary and one or more different temperature ranges are also contemplated.

In an embodiment, the first shape can be a non-linear shape, such as but not limited to a curved, arcuate, coil (including one or more rotations), spiral, helix shape, or the like and the second shape can be substantially linear. In an embodiment, the first shape can be substantially linear, and the second shape can be a non-linear shape such as but not limited to a curved, arcuate, coil (including one or more rotations), spiral, helix shape, or the like.

In an embodiment, the shapeable portion 630 can define a first, non-linear shape at the first temperature, e.g. ambient room temperature or less than 37° C. (98.6° F.). For example, as shown in FIG. 7B, the first shape can a curved shape such that an axis 552 of the distal tip 280 of the stylet 290 extends at an angle (a) relative to a central longitudinal axis 550 of the stylet 290. In an embodiment the angle (a) can be between 1° and 180° relative to the central longitudinal axis 550. However, greater or lesser angles (a) are also contemplated. When placed within the vasculature, the body temperature of the patient, e.g. 37° C. (98.6° F.), can transition the shapeable portion 630 to the second shape, e.g. substantially linear (FIG. 7A).

In an embodiment, the shapeable portion 630 can define a first, substantially linear shape (FIG. 7A) at the first temperature, e.g. ambient room temperature or less than 37° C. (98.6° F.). When placed within the vasculature, the body temperature of the patient, e.g. 37° C. (98.6° F.), can transition the shapeable portion 630 to the second shape, e.g. non-linear (FIG. 7B).

In an embodiment, the shapeable portion 630 can define a relatively rigid state at a first temperature and a relatively flexible state at a second temperature. In an embodiment, the shapeable portion 630 can define a relatively flexible state at a first temperature and a relatively rigid state at a second temperature.

As shown in FIG. 7A, in an embodiment, the shapeable portion 630 can form part of the stylet 290. The shapeable portion 630 can extend annularly about the central axis 550 and can be disposed on an outer surface of the optical fiber 135. Optionally, an insulating layer 210 can extend over the shapeable portion 630 and/or between the shapeable portion 630 and the optical fiber 135. In an embodiment, the shapeable portion 630 can define a part of a lumen extending through the elongate medical device 130. In an embodiment, the shapeable portion 630 can be formed as a layer of shape-memory, super-elastic material disposed on an inner surface of the lumen.

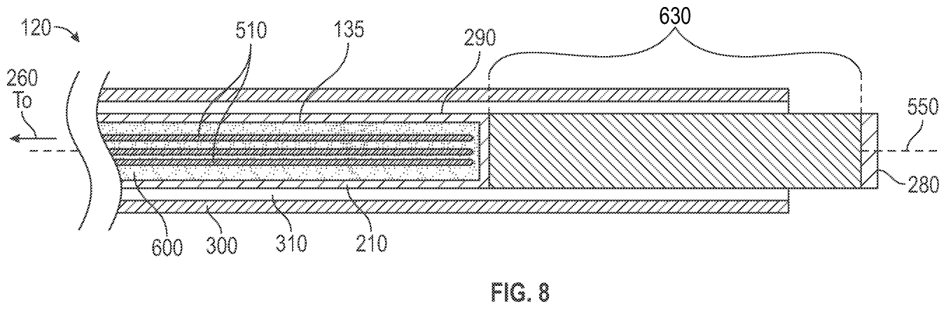

In an embodiment, the shapeable portion 630 can be disposed proximally of a distal tip 280 of the stylet 290. In an embodiment, the shapeable portion 630 can extend to a distal tip 280 of the stylet 290. In an embodiment, as shown in FIG. 8, the shapeable portion can be disposed distally of a distal tip of the optical fiber 135 and can form a distal portion of the stylet 290.

In an embodiment, as shown in FIGS. 7A-7B, the stylet 290 including the shapeable portion 630 can be disposed within a lumen 310 of the catheter body 300. The catheter body 300 can define a relatively more flexible mechanical properties than that of the stylet 290 and/or the shapeable portion 630. As such, as the shapeable portion 630 transitions between the first shape and the second shape, the stylet 290 can flexibly deform a portion of the catheter body 300.

In an embodiment, a user can actuate the steering control system 260 to pass a fluid 82 through a lumen 310 of the catheter body 300 over the stylet 290. The steering control system 260 can include a temperature regulation device 268 configured to modify a temperature and/or flow rate of the fluid 82 being passed through the catheter lumen 310. Exemplary temperature regulation devices 268 can include, but not limited to a heat source, cooling source, thermoelectric generator, Seebeck generator, heat pump, refrigeration system, immersion heater, induction heater, infrared heater, combinations thereof, or the like. Exemplary fluids 82 can include, but not limited to, water, saline, salt solutions, sugar solutions, Ringers solution, blood, plasma, combinations thereof, or the like.

In an embodiment, a temperature of the fluid 82 can modify a shape and/or flexibility of the shapeable portion 630. For example, the temperature of the fluid 82 can be a first relatively cooler temperature, a second relatively warmer temperature, or at a transition temperature therebetween. In an embodiment, the stylet 290 can be at the first shape, or first flexibility outside of the vasculature. As the stylet 290 is advanced into the vasculature, a temperature of the fluid 82 disposed within the lumen 310 can maintain the stylet 290 at the first shape and/or first flexibility despite the ambient temperature surrounding the stylet 290 increasing. In an embodiment, the steering control system 260 can modify the temperature of the fluid 82 to a second temperature, e.g. a temperature that is equal to the temperature within the vasculature. As such, the shapeable portion 630 can transition to a second shape and/or second flexibility state, different from the first flexibility state (i.e. relatively more flexible or less flexible).

In an embodiment, the steering control system 260 can modify the temperature of the fluid 82 to a transition temperature, e.g. a temperature that is between the first temperature and the second temperature. As such, the shapeable portion 630 can transition to a transition shape, different from both the first shape and the second shape, and/or transition to a transition flexibility state, different from both the first flexibility state (e.g. more flexible) and the second flexibility state (e.g. more rigid).

For example, as shown in FIG. 7A, a first shape can define a linear shape and/or define a relatively rigid state. As the stylet 290 enters the vasculature, the temperature of the fluid 82 can maintain the shapeable portion 630 in the linear shape and/or relatively rigid state. The steering control system 260 can then modify a temperature of the fluid 82 to a second temperature and transition the shapeable portion 630 to a non-linear shape, e.g. an angle (a) of 150°. The steering control system 260 can then modify a temperature of the fluid 82 to a second temperature and transition the shapeable portion 630 to second flexibility state, e.g. a relatively flexible state.

In an embodiment, the steering control system 260 can modify a temperature of the fluid 82 to a transition temperature, between the first temperature and the second temperature, and transition the shapeable portion 630 to a transition shape, e.g. an angle (a) of between 1° and 149°. As will be appreciated, these angles are exemplary and not intended to be limiting. In an embodiment, the steering control system 260 can modify a temperature of the fluid 82 to a transition temperature, between the first temperature and the second temperature, and transition the shapeable portion 630 to a transition flexibility state, e.g. more flexible relative to the first (relatively rigid) flexibility state, and less flexible than the second (relatively more flexible) flexibility state.

Advantageously, the steering control system 260 can modify a shape of the shapeable portion 630 to modify an axis 552 of the distal tip 280 relative to the central longitudinal axis 550. Alternatively, or in addition to, the steering control system 260 can modify a flexibility of the shapeable portion 630. Together with manipulating a longitudinal movement and/or a rotational movement of the stylet 290 the user can steer the stylet 290 through tortuous vascular pathways.

As will be appreciated the curved shape shown in FIGS. 7A-7B is exemplary and other non-linear shapes of the first shape, second shape, or transition shape are contemplated. For example, the first shape can be linear and the second shape can be a coiled shape where the shapeable portion extends through one or more 360° rotations. Further the transition shape can extend through less degrees of rotation than the second shape. Similarly, the non-linear shape can define a helical shape where the shapeable portion extends through one or more rotations about the central longitudinal axis 550 and where the transition shape can extend through less degrees of rotation than the second shape. These and other non-linear shapes, or combinations of non-linear shapes are contemplated to fall within the scope of the present invention.

In an embodiment, the stylet 290 can include two or more shapeable portions 630 disposed at different positions along a longitudinal length of the stylet 290 and each configured to modify one or both of a shape and/or flexibility state in response to different temperature ranges. As such, the steering control system 260 can modify one or both of a shape and flexibility state of the stylet 290 at one or more inflexion points along a longitudinal length in response to different temperatures of fluids 82.

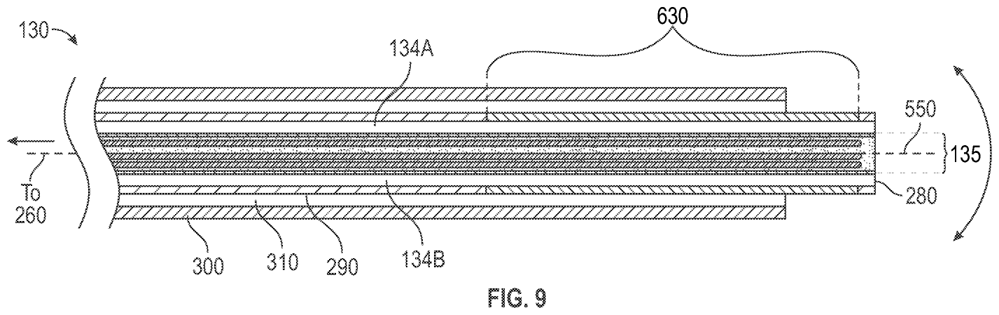

In an embodiment, as shown in FIG. 9, the elongate medical device 130 can include a lumen 134 in fluid communication with the steering control system 260 and through which a temperature regulated fluid 82 can pass. The shapeable portion 630 can form part of the lumen 134 and the shape and/or flexibility of the shapeable portion 630 can be modified by the temperature of the fluid 82 passing through the lumen 134, as described herein.

In an embodiment, as shown in FIG. 9, the elongate medical device 130 can include a first lumen 134A and a second lumen 134B, each extending along a longitudinal axis 550. The steering control system 260 can be configured to provide a fluid 82 to each of the first lumen 134A and the second lumen 134B. The temperature of the fluid 82 within the first lumen 134A can be modified independently of the temperature of the fluid 82 within the second lumen 134B. Advantageously, the stylet 290 can be steered (modify a shape and/or flexibility) in a first direction from the longitudinal axis 550 by modifying a temperature in the first lumen 134A, and can be steered (modify a shape and/or flexibility) in a second direction from the longitudinal axis 550 by modifying a temperature in the second lumen 134B. As will be appreciated, additional lumen 134 can steer the stylet 290 in additional directions relative to the longitudinal axis 550. In an embodiment, one or more of the first lumen 134A, second lumen 134B, etc. can co-ordinate to steer the stylet 290 in additional directions relative to the longitudinal axis 550.

In an exemplary method of use an elongate medical device such as a fiber-optical enabled stylet 290 can be provided including a shapeable portion 630, as described herein. Prior to insertion within a vasculature, the stylet 290 can define a first shape, e.g. a linear shape, at a first temperature. Once the shapeable portion 630 is disposed within the vasculature, the internal body heat of the patient (i.e. equal to or greater than 37° C. (98.6° F.)) can transition the shapeable portion to a second shape, e.g. a non-linear shape. In an embodiment, the second shape can be a curved shape wherein an axis 552 of a distal tip 280 of the stylet 290 extends at an angle (a) relative a central longitudinal axis 550 of the stylet 290.

In an embodiment, the stylet 290 can be advanced through the vasculature to a target location. The steering control system 260 and shapeable portion 630 can be configured to steer a distal portion of the stylet 290 to facilitate negotiating the tortuous vascular pathways. Further, the optical fiber 135 and associated console 110 can be configured to map a pathway through the vasculature and track a location of the stylet 290 relative to the target location. Additional systems such as TLS and ECG tip confirmation systems can also be used to track and confirm the location of the stylet 290 relative to the target location. In an embodiment, once the stylet 290 has reached the target location a second elongate medical device, e.g. catheter assembly 195, can be advanced over the stylet 290 to the target location. In an embodiment, the stylet 290 can be placed within a lumen of a second elongate medical device, e.g. catheter assembly 195, and the stylet 290 and catheter assembly 195 assembly can be advanced concurrently to the target location, with the shapeable portion 630 modifying both the stylet distal tip 280 and the catheter distal tip 360.

In an embodiment, the steering control system 260 can modify a temperature of fluid 82 passing through the catheter lumen 310 (or stylet lumen 134) to modify a shape and/or flexibility of the shapeable portion 630 between, the first shape, second shape, or a transition shape therebetween. Once the shapeable portion 630 has reached the desired shape, e.g. an angle (α), a user can manipulate the handle 240 to rotate the stylet 290 in a desired direction before advancing the stylet longitudinally. In an embodiment, the user can manipulate a flexibility state of the shapeable portion 630 between a relatively rigid state and a relatively flexible to facilitate negotiating tortuous vascular pathways. The user can then observe information about the location of the stylet 290 on the console display 170 from one or more of the FOSS system 100, TLS system, and or ECG tip confirmation system.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A fiber-optic enabled intravascular system, comprising:
a catheter defining a lumen extending along a central longitudinal axis;
an elongate medical device disposed within the catheter lumen and including a shapeable portion, the shapeable portion including a shape-memory, super-elastic material, the shapeable portion defining one or both of a first shape and a first flexibility state at a first temperature and one or both of a second shape and a second flexibility state at a second temperature, which is greater than the first temperature.

2. The fiber-optic enabled intravascular system according to claim 1, further including a steering control system configured to provide a fluid to the catheter lumen and modify a temperature of the fluid to modify a temperature of the shapeable portion between the first temperature and the second temperature.

3. The fiber-optic enabled intravascular system according to claim 2, wherein the steering control system includes one or more of a handle, a fluid source, a pump, and a temperature regulation device configured to modify the temperature of the fluid.

4. The fiber-optic enabled intravascular system according to claim 3, wherein the temperature regulation device includes one or more of heat source, a cooling source, a thermoelectric generator, a Seebeck generator, a heat pump, a refrigeration system, an immersion heater, an induction heater, and an infrared heater.

5. The fiber-optic enabled intravascular system according to claim 1, wherein the elongate medical device includes one of a stylet, trocar, guidewire, or catheter.

6. The fiber-optic enabled intravascular system according to claim 1, wherein the elongate medical device is formed of a first material and the shapeable portion is formed of a second material, the first material including a plastic, polymer, metal, alloy, or composite, the second material including a metal, alloy, shape-memory material, super-elastic material, or Nitinol.

7. The fiber-optic enabled intravascular system according to claim 1, wherein the elongate medical device further includes an optical fiber extending longitudinally and communicatively coupled to a fiber optic strain sensor system configured to determine a shape of the elongate medical device.

8. The fiber-optic enabled intravascular system according to claim 7, wherein the shapeable portion extends annularly about a portion of the optical fiber.

9. The fiber-optic enabled intravascular system according to claim 7, wherein the shapeable portion is disposed distally of a distal tip of the optical fiber.

10. The fiber-optic enabled intravascular system according to claim 1, further including one or both of a transition shape and a transition flexibility state at a third temperature, which is between the first temperature and the second temperature.

11. The fiber-optic enabled intravascular system according to claim 1, wherein the first shape is a linear shape and the second shape is a non-linear shape.

12. The fiber-optic enabled intravascular system according to claim 1, wherein the first shape is a non-linear shape and the second shape is a linear shape.

13. The fiber-optic enabled intravascular system according to claim 10, wherein the first shape is a linear shape and the second shape is a curved shape where an axis of a distal tip of the elongate medical device extends at a first angle relative to the central longitudinal axis, and a transition shape is a curved shape where an axis of the distal tip of the elongate medical device extends at a second angle relative to the central longitudinal axis, less than the first angle.

14. The fiber-optic enabled intravascular system according to claim 2, wherein the elongate medical device further includes a first lumen, and wherein the steering control system is in fluid communication with the first lumen and configured to modify a temperature of the fluid within the first lumen.

15. The fiber-optic enabled intravascular system according to claim 14, wherein the elongate medical device further includes a second lumen, and wherein the steering control system is configured to modify a temperature of the fluid within the first lumen independently of a temperature of the fluid within the second lumen.

16. A method of placing a catheter within a vasculature, comprising:
advancing a distal tip of an elongate medical device into a vasculature of a patient, the elongate medical device including a shapeable portion and disposed within a lumen of a catheter extending along a central longitudinal axis;
modifying a temperature of a fluid to a first predetermined temperature;
urging the fluid through the catheter lumen;
modifying a temperature of the shapeable portion; and
modifying one or both of a shape and a flexibility state of the shapeable portion.

17. The method according to claim 16, wherein the step of modifying the temperature of the fluid includes modifying one or both of a pump and a temperature regulation device of a steering control system, the temperature regulation device including one of a heat source, a cooling source, a thermoelectric generator, a Seebeck generator, a heat pump, a refrigeration system, an immersion heater, an induction heater, and an infrared heater.

18. The method according to claim 16, wherein the elongate medical device includes one of a stylet, trocar, guidewire, or catheter.

19. The method according to claim 16, wherein the elongate medical device is formed of a first material and the shapeable portion is formed of a second material, the first material including a plastic, polymer, metal, alloy, or composite, the second material including a metal, alloy, shape-memory material, super-elastic material, or Nitinol.

20. The method according to claim 16, wherein the shapeable portion transitions between a first shape at a first temperature, and a second shape at a second temperature, the second temperature being greater than the first temperature, and a transition shape at a third temperature that is between the first temperature and the second temperature.

21. The method according to claim 20, wherein the first shape is a linear shape and the second shape is a curved shape where an axis of a distal tip of the elongate medical device extends at a first angle relative to the central longitudinal axis, and a transition shape is a curved shape where an axis of the distal tip of the elongate medical device extends at a second angle relative to the central longitudinal axis, less than the first angle.

22. The method according to claim 16, wherein the shapeable portion transitions between a first flexibility state at a first temperature, and a second flexibility state at a second temperature, the second temperature being greater than the first temperature, and a transition flexibility state at a third temperature that is between the first temperature and the second temperature.

23. The method according to claim 16, wherein the elongate medical device further includes an optical fiber extending therethrough and communicatively coupled to a fiber optic strain sensor system configured to determine a shape of the elongate medical device.

24. A method of placing a fiber-optic enabled medical device within a vasculature, comprising:
providing a the fiber-optic enabled medical device including a shapeable portion at a first temperature, the shapeable portion formed of a shape-memory material;
advancing a distal tip of the fiber-optic enabled medical device into a vasculature of a patient;
modifying a temperature of the shapeable portion from the first temperature to a second temperature, greater than the first temperature; and
modifying one or both of a shape and a flexibility state of the shapeable portion.
